(19) United States
(12) Patent Application Publication
(10) Pub. No.: US 2025/0152234 A1
(43) Pub. Date: May 15, 2025

(54) ELECTROSURGICAL SEAL AND DISSECTION SYSTEMS

(71) Applicant: Applied Medical Resources Corporation, Rancho Santa Margarita, CA (US)

(72) Inventors: Daniel McFarland, Dana Point, CA (US); Michael Whitlock, Irvine, CA (US); Patrick Elliott, Rancho Santa Margarita, CA (US); Duy Nguyen, Rancho Santa Margarita, CA (US); Aaron Jimenez, Eastvale, CA (US); Andrea Chan, Rancho Santa Margarita, CA (US); Vanna Lee, Trabuco Canyon, CA (US)

(21) Appl. No.: 19/024,522

(22) Filed: Jan. 16, 2025

Related U.S. Application Data
(63) Continuation of application No. 17/013,052, filed on Sep. 4, 2020, now Pat. No. 12,239,359.
Application No. 17/013,052 is a continuation of application No. 15/334,102, filed on Oct. 25, 2016, now Pat. No. 10,792,092.
Application No. 15/334,102 is a continuation of application No. PCT/US2015/033546, filed on Jun. 1, 2015.
(60) Provisional application No. 62/004,980, filed on May 30, 2014.
(60) Provisional application No. 62/005,009, filed on May 30, 2014.

(51) Int. Cl. A61B 18/14; A61B 18/00; A61B 18/12; A61B 90/00

(52) U.S. Cl. CPC A61B 18/1445; A61B 18/1206; A61B 18/1233; A61B 2018/00607; A61B 2018/00702; A61B 2018/0075; A61B 2018/00767; A61B 2018/00827; A61B 2018/00869; A61B 2018/00875; A61B 2018/00892; A61B 2018/1452; A61B 2090/0818

(57) ABSTRACT

A bipolar electrosurgical fusion/sealer and dissector is provided that is arranged to simultaneously fuse and cut tissue captured between jaws of the instrument. The jaws include particularly positioned, shaped and/or oriented electrodes along with a compressible landing pad to perform the simultaneous fusion and cutting of tissue. An electrosurgical generator is arranged to supply RF energy through the instrument and monitors a phase angle of the supplied RF energy and adjusts or terminates the supplied RF energy based on the monitored phase angle to optimally fuse and dissect the tissue.

### CROSS-REFERENCE TO RELATED APPLICATIONS

This application is a continuation of U.S. patent application Ser. No. 17/013,052, filed Sep. 4, 2020, which is a continuation of U.S. patent application Ser. No. 15/334,102, filed Oct. 25, 2016, now issued U.S. Pat. No. 10,792,092, which is a continuation of International Application No. PCT/US2015/033546, filed Jun. 1, 2015, which claims the benefit of U.S. Provisional Application No. 62/005,009, filed May 30, 2014, and U.S. Provisional Application No. 62/004,980, filed May 30, 2014, the entire disclosures of which are incorporated by reference as if set forth in full herein.

### BACKGROUND

The present application relates generally to electrosurgical systems and methods and more particularly relates to an electrosurgical fusion/seal and dissection systems.

Electrosurgical devices or instruments have become available that use electrical energy to perform certain surgical tasks. Typically, electrosurgical instruments are surgical instruments such as graspers, scissors, tweezers, blades, needles that include one or more electrodes that are configured to be supplied with electrical energy from an electrosurgical generator. The electrical energy can be used to coagulate, fuse, or cut tissue to which it is applied.

Electrosurgical instruments typically fall within two classifications: monopolar and bipolar. In monopolar instruments, electrical energy is supplied to one or more electrodes on the instrument with high current density while a separate return electrode is electrically coupled to a patient and is often designed to minimize current density. Monopolar electrosurgical instruments can be useful in certain procedures, but can include a risk of certain types of patient injuries such as electrical burns often at least partially attributable to functioning of the return electrode. In bipolar electrosurgical instruments, one or more electrodes is electrically coupled to a source of electrical energy of a first polarity and one or more other electrodes is electrically coupled to a source of electrical energy of a second polarity opposite the first polarity. Bipolar electrosurgical instruments, which operate without separate return electrodes, can deliver electrical signals to a focused tissue area with reduced risks.

Even with the relatively focused surgical effects of bipolar electrosurgical instruments, however, surgical outcomes are often highly dependent on surgeon skill. For example, thermal tissue damage and necrosis can occur in instances where electrical energy is delivered for a relatively long duration or where a relatively high-powered electrical signal is delivered even for a short duration. The rate at which a tissue will achieve the desired coagulation or cutting effect upon the application of electrical energy varies based on the tissue type and can also vary based on pressure applied to the tissue by an electrosurgical device. However, it can be difficult for a surgeon to assess how quickly a mass of combined tissue types grasped in an electrosurgical instrument will be fused a desirable amount.

### SUMMARY

In accordance with various embodiments, an electrosurgical laparoscopic fusion/sealer and dissector instrument is provided that is configured to simultaneously fuse and cut tissue. In various embodiments, the electrosurgical device or instrument includes a first jaw and a second jaw opposing the first jaw to grasp tissue between the first and second jaws. The first jaw includes an electrode and the second jaw includes an electrode. The electrodes of the first and second jaws are arranged to fuse and cut tissue between the first and second jaws using radio frequency energy with center portions of the first and second jaws facing each other being devoid of an electrode.

In various embodiments, an electrosurgical instrument comprises a first jaw with a first electrode having a first surface area to contact tissue and a second electrode with a second surface area to contact tissue. The first surface area is the equal to the second surface area. The instrument also includes a second jaw opposing the first jaws and coupled to the first jaw to grasp tissue between the first and second jaws. The second jaw includes a third electrode having a third surface area to contact tissue and a fourth electrode having a fourth surface area to contact tissue. The third surface area is equal to the fourth surface area and the fourth surface area is greater than the first surface area. The first and third electrodes are arranged to fuse tissue between the first and second jaws using radio frequency energy on one side of a longitudinal axis and the second and fourth electrodes are arranged to fuse tissue between the first and second jaws using radio frequency energy on an opposing side of a longitudinal axis.

In accordance with various embodiments, an electrosurgical system for simultaneously fusing and cutting tissue is provided. The system in various embodiments comprises an electrosurgical generator and an electrosurgical fusion/sealer and dissector instrument or device. The generator includes an RF amplifier and a controller. The RF amplifier supplies RF energy through a removably coupled electrosurgical instrument, e.g., an electrosurgical fusion and dissector, configured to fuse and cut tissue with only RF energy. The controller is arranged to monitor a phase angle of the supplied RF energy, the controller signaling the RF amplifier to increase voltage of the supplied RF energy when the monitored phase angle is greater than zero and increasing. In various embodiments, the controller signals the RF amplifier to halt the supplied RF energy when the monitored phase angle decreases.

Many of the attendant features of the present inventions will be more readily appreciated as the same becomes better understood by reference to the foregoing and following description and considered in connection with the accompanying drawings.

### BRIEF DESCRIPTION OF THE DRAWINGS

The present inventions may be better understood taken in connection with the accompanying drawings in which the reference numerals designate like parts throughout the figures thereof.

FIG. 1 is a perspective view of an electrosurgical system in accordance with various embodiments of the present invention.

FIG. 2 is a perspective view of an electrosurgical generator in accordance with various embodiments of the present invention.

FIG. 3 is a perspective view of an electrosurgical instrument in accordance with various embodiments of the present invention.

FIG. 4 is a perspective view of a distal end of the electrosurgical instrument in accordance with various embodiments of the present invention.

FIG. 5 is a perspective view of a distal end of the electrosurgical instrument in accordance with various embodiments of the present invention.

FIG. 6 is a cross-sectional view of a distal end of an electrosurgical instrument in accordance with various embodiments of the present invention.

FIG. 7 is a cross-sectional view of a distal end of an electrosurgical instrument in accordance with various embodiments of the present invention.

FIG. 8 is a cross-sectional view of a distal end of an electrosurgical instrument in accordance with various embodiments of the present invention.

FIG. 9 is a cross-sectional view of a distal end of an electrosurgical instrument in accordance with various embodiments of the present invention.

FIG. 10 is a cross-sectional view of a distal end of an electrosurgical instrument in accordance with various embodiments of the present invention.

FIG. 11 is a graphical representation of samples of experimental data for a fusion and dissection process with an electrosurgical instrument in accordance with various embodiments of the present invention.

FIG. 12 is a graphical representation of samples of experimental data for a fusion and dissection process with an electrosurgical instrument in accordance with various embodiments of the present invention.

FIG. 13 is a graphical representation of samples of experimental data for a fusion and dissection process with an electrosurgical instrument in accordance with various embodiments of the present invention.

FIG. 14 is a cross-sectional view of a distal end of an electrosurgical instrument in accordance with various embodiments of the present invention.

FIG. 15 is a flow chart illustrating operations of an electrosurgical system in accordance with various embodiments of the present invention.

FIG. 16 is a schematic block diagram of portions of an electrosurgical system in accordance with various embodiments of the present invention.

FIG. 17 is a schematic block diagram of portions of an electrosurgical system in accordance with various embodiments of the present invention.

FIG. 18 is a schematic block diagram of portions of an electrosurgical system in accordance with various embodiments of the present invention.

FIG. 19 is a flow chart illustrating operations of an electrosurgical system in accordance with various embodiments of the present invention.

FIG. 20 is a flow chart illustrating operations of an electrosurgical system in accordance with various embodiments of the present invention.

FIG. 21 is a flow chart illustrating operations of an electrosurgical system in accordance with various embodiments of the present invention.

FIG. 22 is a graphical representation of samples of experimental data for an electrosurgical system in accordance with various embodiments of the present invention.

FIG. 23 is a graphical representation of samples of experimental data for an electrosurgical system in accordance with various embodiments of the present invention.

FIG. 24 is a graphical representation of samples of experimental data for an electrosurgical system in accordance with various embodiments of the present invention.

### DETAILED DESCRIPTION

Generally, a bipolar electrosurgical fusion/sealer and dissector instrument, device or tool is provided that is arranged to simultaneously fuse and cut tissue captured between jaws of the instrument. The jaws include particularly positioned, shaped and/or oriented electrodes along with a compressible landing pad to optimally perform the simultaneous fusion and cutting of tissue. The bipolar electrosurgical fusion and dissector can also separately fuse or cut tissue. The cutting of tissue in accordance with various embodiments is notably performed without the use of a mechanical cutting blade, the use of a particular or center cut electrode or the shearing forces or movement of a scissor. The instrument in accordance with various embodiments is provided to be used in laparoscopic surgery having a maximum diameter of 5 mm and thus is insertable through a 5 mm trocar.

Additionally, in general, an electrosurgical system is provided that includes an electrosurgical generator and a removably coupled electrosurgical instrument, e.g., a fusion and dissector, that are configured to optimally fuse and cut tissue. The RF energy is supplied by the electrosurgical generator that is arranged to provide the appropriate RF energy to fuse and cut the tissue. The generator in accordance with various embodiments determines the appropriate RF energy and the appropriate manner to deliver the RF energy for the particular connected electrosurgical instrument, the particular tissue in contact with the instrument and/or a particular surgical procedure. Operationally, RF sealing or fusing of tissue between the jaws is provided to decrease sealing time, output voltage, output power and/or thermal spread. As such, efficiently and consistently delivering power to tissue is provided to heat tissue through a range of temperatures at a particular rate that has been found to be optimal for the tissue affect.

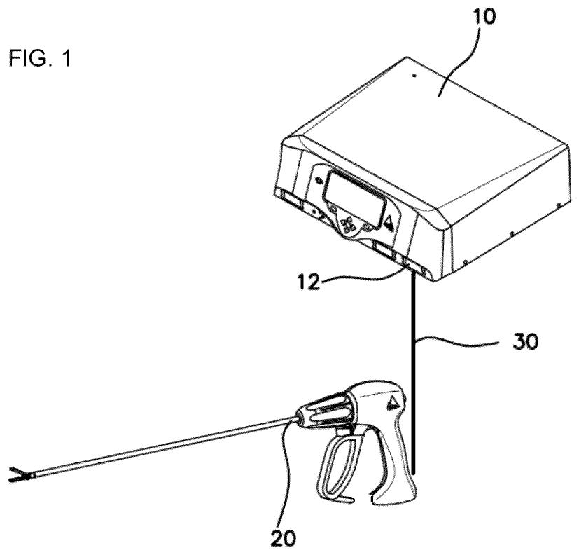

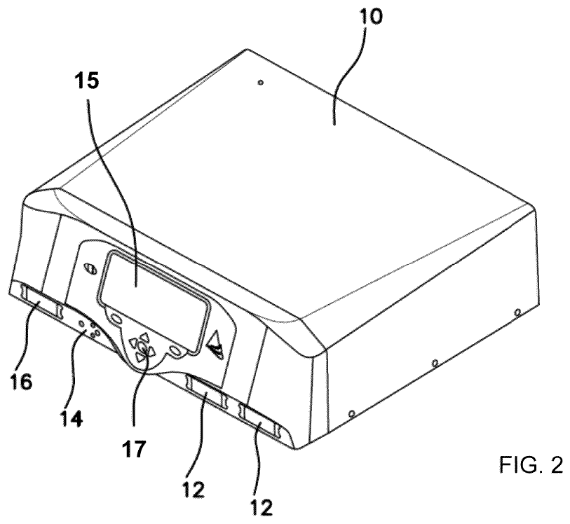

Referring to FIGS. 1-2, an exemplary embodiment of electrosurgical system is illustrated including an electrosurgical generator 10 and a removably connectable electrosurgical instrument 20. The electrosurgical instrument 20 can be electrically coupled to the generator via a cabled connection 30 to a tool or device port 12 on the generator. The electrosurgical instrument 20 may include audio, tactile and/or visual indicators to apprise a user of a particular predetermined status of the instrument such as a start and/or end of a fusion or cut operation. In other embodiments, the electrosurgical instrument 20 can be reusable and/or connectable to another electrosurgical generator for another surgical procedure. In some embodiments, a manual controller such as a hand or foot switch can be connectable to the generator and/or instrument to allow predetermined selective control of the instrument such as to commence a fusion or cut operation.

In accordance with various embodiments, the electrosurgical generator 10 is configured to generate radiofrequency (RF) electrosurgical energy and to receive data or information from the electrosurgical instrument 20 electrically coupled to the generator. The generator 10 in one embodiment outputs RF energy (e.g., 375 VA, 150V, 5 A at 350 kHz) and in one embodiment is configured to calculate a phase angle or difference between RF output voltage and RF output current during activation or supply of RF energy. The generator regulates voltage, current and/or power and monitors RF energy output (e.g., voltage, current, power and/or phase). In one embodiment, the generator 10 stops RF energy output under predefine conditions such as when a device switch is de-asserted (e.g., fuse button released), a time value is met, and/or active phase angle and/or change of phase is greater than or equal to a phase and/or change of phase stop value.

The electrosurgical generator 10 comprises two advanced bipolar tool ports 12, a standard bipolar tool port 16, and an electrical power port 14. In other embodiments, electrosurgical units can comprise different numbers of ports. For example, in some embodiments, an electrosurgical generator can comprise more or fewer than two advanced bipolar tool ports, more or fewer than the standard bipolar tool port, and more or fewer than the power port. In one embodiment, the electrosurgical generator comprises only two advanced bipolar tool ports.

In accordance with various embodiments, each advanced bipolar tool port 12 is configured to be coupled to electrosurgical instrument having an attached or integrated memory module. The standard bipolar tool port 16 is configured to receive a non-specialized bipolar electrosurgical tool that differs from the advanced bipolar electrosurgical instrument connectable to the advanced bipolar tool port 12. The electrical power port 14 is configured to receive or be connected to a direct current (DC) accessory device that differs from the non-specialized bipolar electrosurgical tool and the advanced electrosurgical instrument. The electrical power port 14 is configured to supply direct current voltage. For example, in some embodiments, the power port 14 can provide approximately 12 Volts DC. The power port 14 can be configured to power a surgical accessory, such as a respirator, pump, light, or another surgical accessory. Thus, in addition to replacing electrosurgical generator for standard or non-specialized bipolar tools, the electrosurgical generator can also replace a surgical accessory power supply. In some embodiments, replacing presently-existing generators and power supplies with the electrosurgical generator can reduce the amount of storage space required on storage racks cards or shelves in the number of mains power cords required in a surgical workspace.

In one embodiment, connection of a non-specialized bipolar tool into the standard bipolar port will not cause the generator to actively check the tool. However, the generator recognizes a connection so that the information of the non-specialized bipolar tool can be displayed. In accordance with various embodiments, the generator recognizes device connection status for each of the advanced tool ports 12 and authenticates connected devices before accepting RF energy activation requests (e.g., activation of an instrument switch such as a fuse button). The generator in one embodiment reads authenticated data from the connected device and reads electrical control values (such as but not limited to voltage level settings, current level settings, power level settings, active phase angle level settings, RF energy output activation timing limits, instrument short limits, instrument open limits, instrument model/identification, RF energy output line configurations, switch state command configurations and/or combinations thereof) from the authenticated and connected device.

In accordance with various embodiments, the electrosurgical generator 10 can comprise a display 15. The display can be configured to indicate the status of the electrosurgical system including, among other information, the status of the one or more electrosurgical instruments and/or accessories, connectors or connections thereto. In some embodiments, the display can comprise a multi-line display capable of presenting text and graphical information such as for example an LCD panel display, which, in some embodiments can be illuminated via backlight or sidelight. In some embodiments, the display can comprise a multi-color display that can be configured to display information about a particular instrument electrically coupled to the electrosurgical generator and a color that corresponds to a particular surgical procedure (such as, for example cutting operations displayed in yellow text and graphics, fusion or welding operations displayed in purple, and coagulation displayed in blue, bloodless dissection operations can be displayed in yellow and blue).

In some embodiments, the display can be configured to simultaneously indicate status data for a plurality of instruments electrically coupled to the electrosurgical generator and/or be portioned to display status information for each instrument connected to a corresponding tool port. A visual indicator such as a status bar graph can be used to illustrate a proportion of total available electrical energy to be applied to the bipolar electrosurgical instrument when actuated. In various embodiments, an electrosurgical instrument operable to cut, seal, coagulate, or fuse tissue could have three color-coded displays or bar graphs. In some embodiments, a user can toggle the display between presenting status of multiple electrically connected instruments and status of a single electrically connected instrument. In accordance with various embodiments, once an instrument and/or accessory is connected and/or detected a window opens in the user interface display showing the type of instrument connected and status.

The electrosurgical generator in accordance with various embodiments can comprise a user interface such as, for example a plurality of buttons 17. The buttons can allow user interaction with the electrosurgical generator such as, for example, requesting an increase or decrease in the electrical energy supplied to one or more instruments coupled to the electrosurgical generator. In other embodiments, the display 15 can be a touch screen display thus integrating data display and user interface functionalities. In accordance with various embodiments, through the user interface, the surgeon can set a voltage setting by the selection of one to three levels. For example, at level 1, voltage is set to 110V; at level 2, voltage is set to 100V; and at level 3, voltage is set to 90V. Current is set to 5 Amps and power is set to 300 VA for all three levels. In other embodiments, the voltage is preset or defaults to a specific level such as level 2. In other embodiments, like the current and power settings, the voltage setting is not user adjustable to simplify operation of the generator and as such a predetermined default voltage setting is utilized, e.g., voltage is set to 100V.

In one embodiment, the electrosurgical tool or instrument 20 can further comprise of one or more memory modules. In some embodiments, the memory comprises operational data concerning the instrument and/or other instruments. For example, in some embodiments, the operational data may include information regarding electrode configuration/reconfiguration, the instrument uses, operational time, voltage, power, phase and/or current settings, and/or particular operational states, conditions, scripts, processes or procedures. In one embodiment, the generator initiate reads and/or writes to the memory module.

In one embodiment, each advanced bipolar electrosurgical instrument comes with a memory module and/or an integrated circuit that provides instrument authentication, configuration, expiration, and logging. Connection of such instruments into the receptacles or ports initiates an instrument verification and identification process. Instrument authentication in one embodiment is provided via a challenge-response scheme and/or a stored secret key also shared by the generator. Other parameters have hash keys for integrity checks. Usages are logged to the generator and/or to the instrument integrated circuit and/or memory. Errors in one embodiment can result in unlogged usage. In one embodiment, the log record is set in binary and interpreted with offline instruments or via the generator.

In one embodiment, the generator uses time measurement components to monitor an instrument's expiration. Such components utilize polling oscillators or timers or real-time calendar clocks that are configured at boot time. Timer interrupts are handled by the generator and can be used by scripts for timeout events. Logging also utilizes timers or counters to timestamp logged events.

In accordance with various embodiments, the generator provides the capability to read the phase difference between the voltage and current of the RF energy sent through the connected electrosurgical instrument while RF energy is active. While tissue is being fused, phase readings are used to detect different states during the fuse or seal and cut process.

In one embodiment, the generator logs usage details in an internal log that is down loadable. The generator has memory for storage of code and machine performance. The generator has reprogrammable memory that contains instructions for specific instrument performance. The memory for example retains a serial number and instrument use parameters. The generator stores information on the type of instruments connected. Such information includes but is not limited to an instrument identifier, e.g., a serial number of a connected instrument, along with a time stamp, number of uses or duration of use of the connected instrument, power setting of each and changes made to the default setting. The memory in one embodiment holds data for about two months, about 10,000 instrument uses or up to 150 logged activations and is configured to overwrite itself as needed.

The generator in accordance with various embodiments does not monitor or control current, power or impedance. The generator regulates voltage and can adjust voltage. Electrosurgical power delivered is a function of applied voltage, current and tissue impedance. The generator through the regulation of voltage can affect the electrosurgical power being delivered. However, by increasing or decreasing voltage, delivered electrosurgical power does not necessarily increase or decrease. Power reactions are caused by the power interacting with the tissue or the state of the tissue without any control by a generator other than by the generator supplying power.

The generator once it starts to deliver electrosurgical power does so continuously, e.g., every 150 ms, until a fault occurs or a specific phase parameter is reached. In one example, the jaws of the electrosurgical instrument can be opened and thus compression relieved at any time before, during and after the application of electrosurgical power. The generator in one embodiment also does not pause or wait a particular duration or a predetermined time delay to commence termination of the electrosurgical energy.

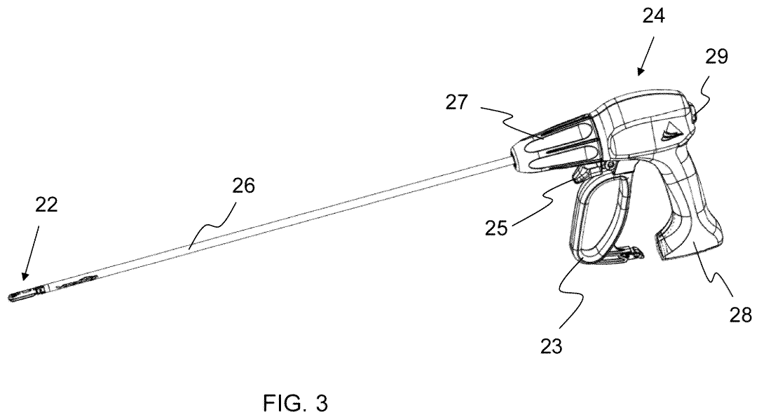

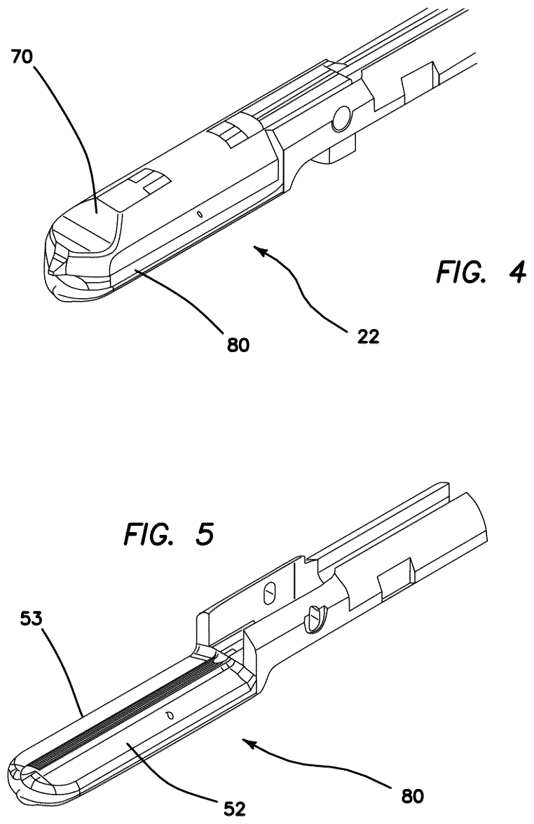

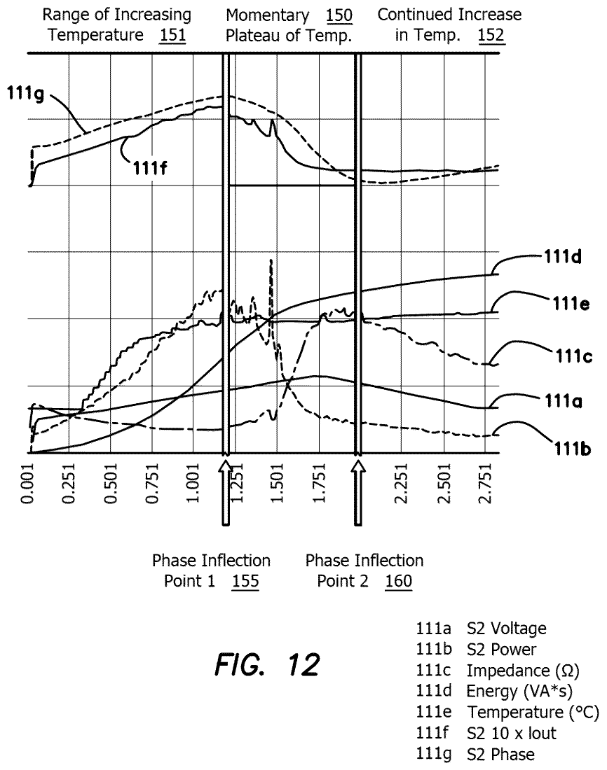

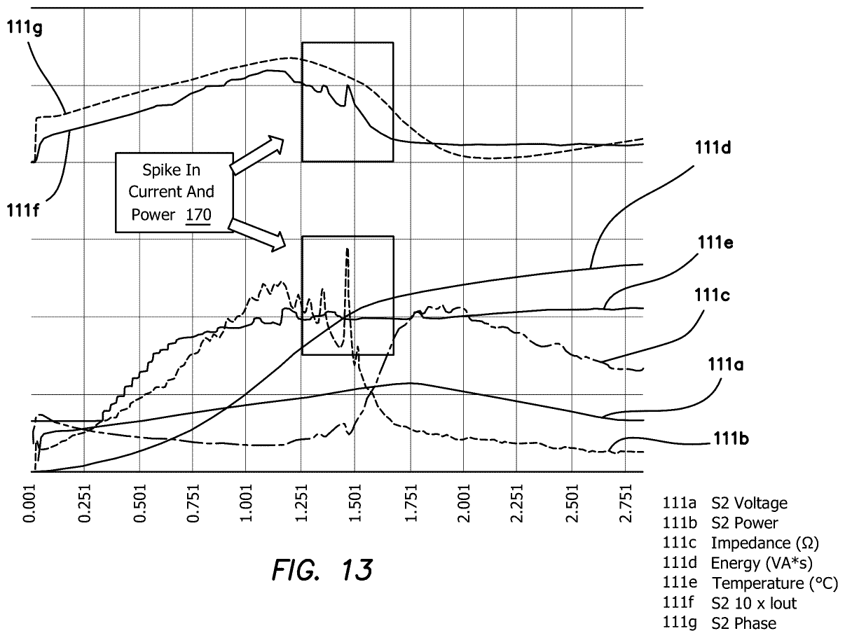

With reference to FIGS. 3-14, in accordance with various embodiments, an bipolar fusion and dissector electrosurgical instrument 20 is provided. In the illustrated embodiment, the instrument 20 includes an actuator 24 coupled to an elongate rotatable shaft 26. The elongate shaft 26 has a proximal end and a distal end defining a central longitudinal axis therebetween. At the distal end of the shaft 26 are jaws 22 and at the proximal end is the actuator. In one embodiment, the actuator is a pistol-grip like handle. The shaft 26 and jaws 22, in one embodiment, are sized and shaped to fit through a 5 mm diameter trocar cannula or access port.

The actuator 24 includes a movable handle 23 and a stationary handle or housing 28 with the movable handle 23 coupled and movable relative to the stationary housing. In accordance with various embodiments, the movable handle 23 is slidably and pivotally coupled to the stationary housing. In operation, the movable handle 23 is manipulated by a user, e.g., a surgeon to actuate the jaws, for example, selectively opening and closing the jaws. In accordance with various embodiments, the actuator 24 includes a force regulation mechanism that is configured such that in a closed configuration, the jaws 22 delivers a gripping force between a predetermined minimum force and a predetermined maximum force.

As part of the force regulation mechanism, the movable handle 23 is coupled to the stationary handle at two sliding pivot locations to form the force regulation mechanism. The movable handle has a first end including a gripping surface formed thereon and a second end opposite the first end. The movable handle is coupled to a pin adjacent the second end. In some embodiments, the movable handle can be integrally formed with a protrusion extending therefrom defining a pin surface, while in other embodiments, a pin can be press-fit into an aperture in the movable handle. The pin can be contained within slots in the stationary housing, such as a corresponding slot formed in a right and/or left handle frames of the stationary housing. In some embodiments, the slots can be configured to define a desired actuation handle path, such as a curved or angled path, as the actuation handle is moved from the first position corresponding to open jaws to a second position corresponding to closed jaws. The force regulation mechanism includes a biasing member such as a tension spring that biases the pin in a proximal direction. In operation, as a predetermined force is exerted on by movement of the movable handle, a biasing force exerted by the spring is overcome, and the second end of the movable handle can translate generally distally, guided by the pin in the slots.

In accordance with various embodiments, the movable handle is slidably and pivotably coupled to the stationary housing 28 at a location between the first and second ends of the actuation handle. An actuation member such as a pull block is coupled to the actuation handle. When the movable handle is moved proximally, the pull block also moves proximally and longitudinally, closing the jaws 22 thereby clamping any tissue between the jaws. The pull block in accordance with various embodiments is rectangular having open top and bottom faces and a closed proximal end. The movable handle extends through the top and bottom faces of the pull block. An edge of the movable handle bears on the proximal end of the pull block such that movement of the movable handle relative to the stationary housing moves the pull block longitudinally. A distal end of the pull block in one embodiment is coupled to an actuation shaft such as a pull tube, bar, or rod, which can extend longitudinally along the elongate shaft 26. Thus, in operation, movement of the movable handle from the first position to the second position translates the pull block longitudinally within the stationary housing, which correspondingly translates the pull tube generally linearly along the longitudinal axis with respect to the elongate shaft 26. Movement of this pull tube can control relative movement of the jaws 22.

In accordance with various embodiments, the actuator 24 includes a latch mechanism to maintain the movable handle 23 in a second position with respect to the stationary housing 28. In various embodiments, the movable handle comprises a latch arm which engages a matching latch contained within stationary handle for holding the movable handle at a second or closed position. The actuator in various embodiments also comprises a wire harness that includes insulated individual electrical wires or leads contained within a single sheath. The wire harness can exit the stationary housing at a lower surface thereof and form part of the cabled connection. The wires within the harness can provide electrical communication between the instrument and the electrosurgical generator and/or accessories thereof.

In accordance with various embodiments, the actuator includes one or more leads attached to rotational coupling clips configured to allow infinite rotation of the shaft. In various embodiments, a switch is connected to a user manipulated activation button 29 and is activated when the activation button is depressed. In one aspect, once activated, the switch completes a circuit by electrically coupling at least two leads together. As such, an electrical path is then established from an electrosurgical generator to the actuator to supply RF energy to the leads attached to the rotational coupling clips.

In one embodiment, the actuator includes a rotation shaft assembly including a rotation knob 27 which is disposed on an outer cover tube of the elongate shaft 26. The rotation knob allows a surgeon to rotate the shaft of the device while gripping the actuator 24. In accordance with various embodiments, the elongate shaft 26 comprises an actuation tube coupling the jaws 22 with the actuator. In various embodiments, the actuation tube is housed within an outer cover tube. While the actuation tube is illustrated as a generally tubular member that can be nested within the outer cover tube, in other embodiments, a non-tubular actuation member can be used, for example, a shaft, a rigid band, or a link, which, in certain embodiments can be positioned within the outer cover tube.

In accordance with various embodiments, attached to the distal end of the outer cover tube is the rotational shaft assembly comprising two mating hubs and a conductive sleeve. The hubs snap together, engaging with the outer cover tube. In other embodiments, the hubs can be of a monolithic construction and configured to interface with mating features on the outer cover tube. The conductive sleeve can be attached to the proximal portion of the assembled hubs after they are attached to the outer cover tube. When the conductive sleeve is attached to the rear of the assembled hubs, the sleeve traps the exposed end of an isolated wire. In the illustrated embodiment, the isolated wire extends from its entrapment point under the conductive sleeve through a slot in the actuation tube and then inside a protective sleeve. The protective sleeve and isolated wire extend distally inside the actuation tube, towards the jaws. In other embodiments, the isolated wire can be formed integrally with a protective sheath and no separate protective sleeve is present in the actuation tube.

Attached to the distal end of the elongate shaft are jaws 22 that comprise a first jaw 70 and a second jaw 80. In one embodiment, a jaw pivot pin pivotally couples the first and second jaws and allows the first jaw to be movable and pivot relative to the second jaw. In various embodiments, one jaw is fixed with respect to the elongate shaft such that the opposing jaw pivots with respect to the fixed jaw between an open and a closed position. In other embodiments, both jaws can be pivotally coupled to the elongate shaft such that both jaws can pivot with respect to each other.

The jaw geometry provides for specific pressure profiles and specific current densities in specific locations to produce the required fusion/seal and dissection effect. Operationally, the temperature required to achieve sealing and division is minimized while the cross linking of proteins within the vascular structure are maximized thereby maximizing the efficacy of the fuse/seal and division of tissue.

In accordance with various embodiments, in order to monitor the temperature of the biological reaction, phase angle and/or the rate of change of the phase angle is monitored. It has been found that the phase angle provides indicators of the temperature of the biological reaction and an indication that the division of the tissue has occurred. In accordance with various embodiments, the device uses bipolar RF energy for electrosurgery for cutting and fusing of tissue between the jaws when opened or closed and/or in contact with the lower jaw when the jaws are open or closed. In one embodiment, temperature of the tissue during the seal and/or division cycle is monitored.

The advanced bipolar electrosurgical device in accordance with various embodiments uses bipolar RF energy for both the sealing or fusing and the division or cutting of tissue. As such, the device maintains cellular structure of tissue adjacent to the area of division while applying the energy required for the division of tissue. Other RF dissection devices use localized arcing or a spark gap to vaporize tissue and achieve dissection. This may be acceptable for straight tissue dissection because the surrounding area is not also intended to be sealed or fused, but is unlike the fusion and dissector devices and systems in accordance with various embodiments of the present invention.

The advanced bipolar electrosurgical device in accordance with various embodiments also accounts for the high heat associated with the vaporization of tissue or tissue dissection. As such, the device in various embodiments uses temperature control to minimize the energy required to achieve tissue division. By minimizing the energy required, the temperature during the reaction is lower and the cellular structure is less likely to be disrupted due to high energy output.

Maintaining the cellular structure of the operating tissue is needed when simultaneously fusing and dissecting or dividing because it is necessary for sealing to occur adjacent to the area of division. The addition of open cut and seal mode also reduces the number of devices or the exchange of devices used in performance of a surgical procedure as such or all such functionality of such individual devices are provided in the single advanced bipolar laparoscopic device.

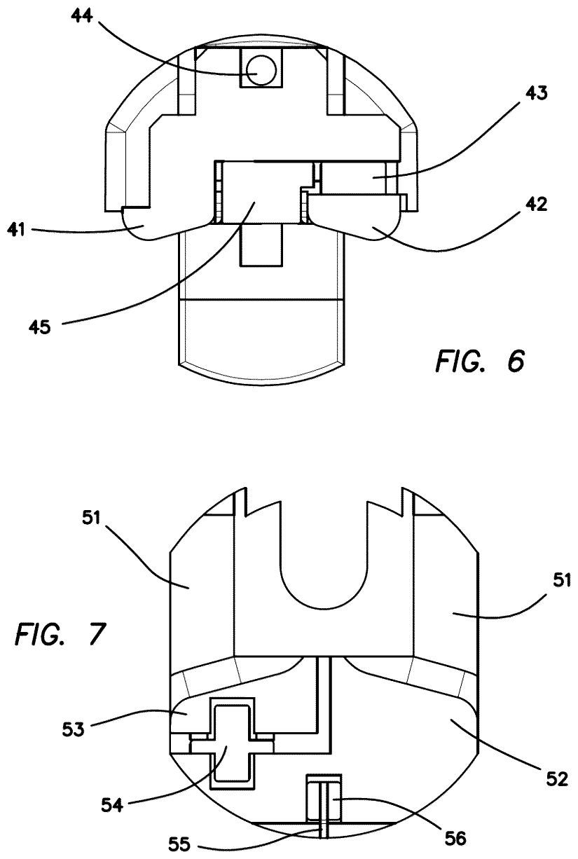

In various embodiments, the electrosurgical instrument comprises movable jaws able to capture tissue therebetween. In one embodiment, the jaws include one upper jaw that closes onto a static lower jaw. In accordance with various embodiments, the upper jaw includes a rigid upper jaw member 41, an upper conductive pad 42, a rigid insulating pad 43, a wire 44, and a compressible landing pad 45 which are all bound together using an insert molding process and thus is provided as a single structure or assembly as shown in the FIG. 6.

The rigid upper jaw member and the upper conductive pad are both active electrodes which have opposite polarities. The compressible landing pad provides a surface with a specific spring rate to ensure that contact and pressure occurs between the landing pad and the length of the lower jaw. The upper conductive pad 42 is electrically isolated from the rigid upper jaw member 41 by the landing pad 45 and the insulating pad 43. In various embodiments, the upper jaw is made of stainless steel and is more rigid than the landing pad 45. In various embodiments, the landing pad 45 is made of silicone and is more compliant than the upper jaw member 41 or conductive pad 42. In various embodiments, the insulating pad is made of a non-conductive material and is as rigid as or more rigid than the upper jaw member 41 or the conductive pad 42. In various embodiments, the upper jaw member 41 and the conductive pad are made of the same material.

The upper jaw member 41 and the conductive pad have a lower outer surface arranged to be in contact with tissue. The lower surfaces are angled or sloped and mirror images of each other with such positioning or orientation facilitating focused current densities and securement of tissue. The compressible landing pad 45 has a lower surface arranged to be in contact with tissue and/or the lower jaw. The landing pad in the illustrated embodiment is flat and non-parallel relative to the sloped lower surfaces of upper jaw member and the conductive pad 42. The positioning or orientation of the lower surface of the landing pad assists in focusing current densities, securing of tissue and facilitating electrically dissecting of tissue. The spring rate of the landing pad in various embodiments is predetermined to provide optimal pressure or force to cause or facilitate the electrical division of tissue.

The lower jaw comprises a rigid lower jaw member 52, a lower conductive pad 53, a cut electrode 55, two rigid insulators 54, 56, as well as two wires, one to the conductive pad and one to the cut electrode, all of which is bound together using an insert molding process and thus are provided as a single structure or assembly as shown in FIG. 7 in accordance with various embodiments of the present invention. The rigid lower jaw member 52, the lower conductive pad 53, and the cut electrode 55 are all or act as active electrodes. The lower conductive pad 53 and the cut electrode 55 have the same polarity and are electrically isolated by the two rigid insulators 54 and 56 from the rigid lower jaw member which has the opposite polarity.

The lower jaw member 52 and the conductive pad 53 have an upper outer surface arranged to be in contact with tissue. The upper surfaces are angled or sloped and mirror images of each other with such positioning or orientation facilitating focused current densities and securement of tissue. In various embodiments, the lower jaw is made of stainless steel and is as rigid as or more rigid than the conductive pad 53. In various embodiments, the rigid insulators 54, 56 are made of a non-conductive material and are as rigid as or more rigid than the lower jaw member 52 or the conductive pad 53. In various embodiments, the lower jaw member 52 and the conductive pad 53 are made of the same material.

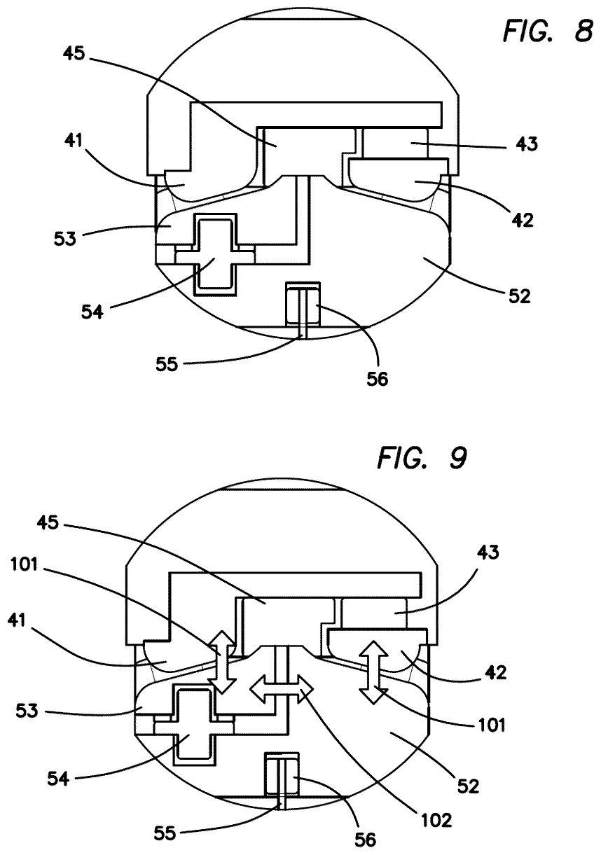

The overall jaw configuration in accordance with various embodiments is shown in cross-section in FIG. 8 and demonstrates the interaction of the geometries (e.g., shape, dimension, material and any combination thereof for optimal fusion and dissection) between the upper jaw and the lower jaw. In operation, the conductive pads 42, 53 are of the same polarity. The upper and lower jaw members 41, 51, 52 are the same polarity, but the opposite polarity of the conductive pads 42, 53. In one embodiment, the cut electrode 55 is only active during an open cut and fuse operation and is the opposite polarity of the lower jaw member 52. As illustrated, the landing pad 45 interferes and compresses onto the lower jaw member 52 and conductive pad 53 upon closure of the jaws. Tissue (not shown) also captured between the lower jaw and the upper jaw is compressed between the landing pad 45 and the lower jaw member 52 and the conductive pad 53.

The polarity of each electrode is set to create the appropriate RF energy and heating due to electrical current passing therebetween. As shown in FIG. 9, the direction of current flow allows for heating between the conductive pads and the jaw members, as well as creating heating from side to side on the lower jaw configuration, as exemplified by arrows 101. The side to side heating on the lower jaw configuration is provided for the division of tissue, as exemplified by arrow 102. In order to divide the tissue down the middle of the jaw, the tissue is heated to reach a temperature between 60° C. and 100° C. to denature the collagen present in the tissue. Once the collagen has been denatured, it enters into a gelatin like state.

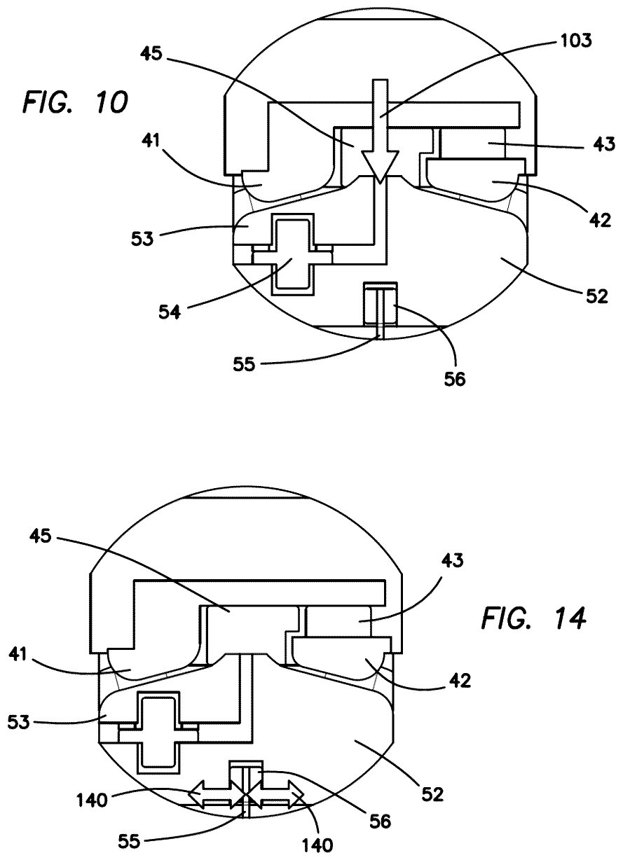

As the tissue is gelatinous or in a gelatin like state, the spring rate and the interference of the silicone landing pad causes mechanical separation of the tissue as exemplified in FIG. 10 and arrow 103. In various embodiments, the spring rate is predetermined to optimize the separation of the tissue by the interference with the pad and the lower jaw such that the landing pad compresses a predetermined distance or amount in conformance with the tissue there between and with minimal or no effects to the adjacent tissue. Therefore, the configuration of the electrodes allow for simultaneous heating of the seal area (the area between the conductive pads and the jaw members) and the cut area (the side to side current region on the lower jaw). The denaturing of the collagen is also the mechanism used to create a tissue seal or fuse. Sealing utilizes the same temperature (60° C. to 100° C.) as cutting, but the jaw seal spacing between the conductive pads and the jaw members creates a form or mold for the seal to re-cross-link once the RF application ceases. It is noted that reaching high temperatures rapidly can cause the cellular structure to rupture due to the rapid heating of intercellular moisture. Therefore, a gradual increase of temperature and a longer dwell time in the appropriate temperature range allows for more thorough denaturing of the collagen.

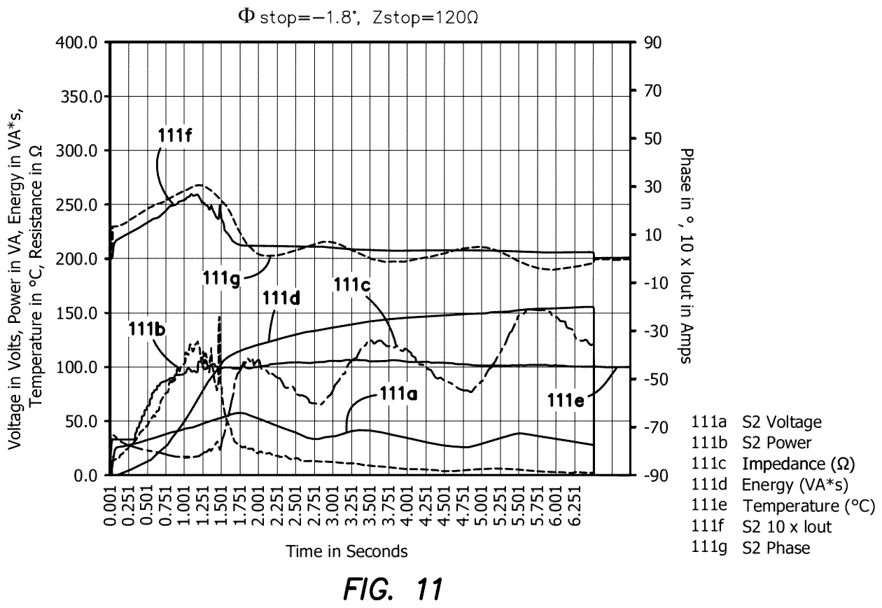

In various embodiments, in order to achieve the appropriate temperature of the tissue to cause the associated tissue effect, e.g., a gradual increase and/or longer dwell time, the phase angle of the tissue and/or the rate of change of the phase angle are monitored. FIGS. 11-13 provide a graphical representation of exemplary seal and division cycles in accordance with various embodiments. Also, as illustrated, phase 111g is shown relative to other tissue readings or indicators such as voltage 111a, power 111b, impedance 111c, energy 111d, temperature 111e and current 111f. Additionally, although shown in FIGS. 11-13, in various embodiments, the generator is configured to not measure or calculate one or more of the indicators or readings, e.g., temperature or energy, to reduce operational and power costs and consumptions and/or the number of parts of the generator. The additional information or readings are generally provided or shown for context purposes.

As shown in FIGS. 11-13, the temperature of the tissue 111e between the jaws increases from the initiation of RF energy to a point of the highest phase angle. At this point of the highest phase angle (or the point of inflection of the rate of change of phase angle) 155 the temperature plateaus 150 momentarily (˜0.75 seconds), then continues higher 152, even though voltage decreases.

Due to the behavior of the temperature profile, it is noted that the momentary plateau in temperature may be attributed to the change in state of the water or moisture present in the tissue. When the water begins to boil, the temperature does not increase until the liquid water has been turned to water vapor. The determination of the temperature 111e as such can be based off the phase angle 111g. The temperature prior to the maximum phase angle is associated with a steady heating to 100° C. The plateau in temperature is associated with a sudden decrease in the phase angle which can be associated with 100° C. and two-state water. Because liquid water is highly conductive and water vapor is not, this transition in phase may be another indicator of the water's state. Once the temperature continues to increase past 100° C. (152) and past a second point of phase angle inflection 160, it can be noted that the majority of the water has been turned to water vapor.

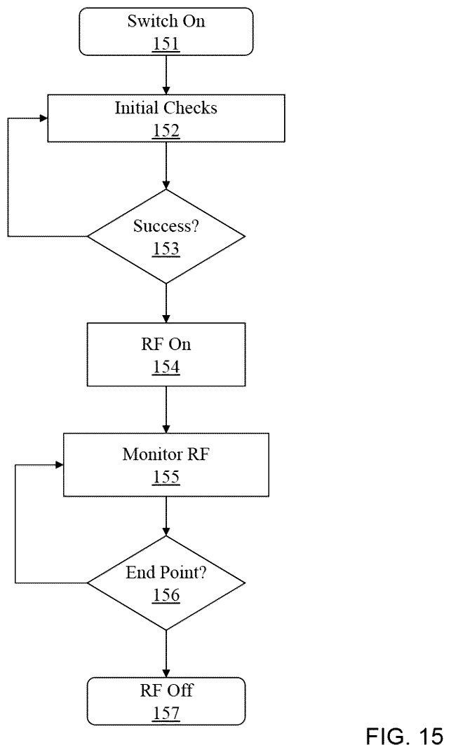

Another point of interest that can be seen in the RF output is the sudden spike 170 in power 111b and current 111f during the boiling of water portion of the RF application as shown for example in FIG. 13. The spike can be attributed to the division of the tissue during the sealing or fusing process. This increase in power and current can be due to tissue being no longer present under the insulated portion of the jaws, e.g., the landing pad. At this point, the jaw closes more, and the energy transfer is only through the seal surfaces.

Since the temperature required to denature the collagen begins at 60° C., the energy application is optimized to maximize the time prior to 100° C. This provides for a complete and thorough denaturing of the collagen. As such, all sealing should be completed prior to the spike 170 in power and current such that sealing is completed prior to division.

In accordance with various embodiments, the electrosurgical instrument also has the ability to cut tissue using RF energy and in one embodiment only with the jaws in a fully open position, only utilizing the lower jaw, without cooperation of the upper jaw or tissue not captured between the upper and lower jaws but in contact with the lower jaw. FIG. 14 shows the direction of current flow, arrow 140, from the cut electrode 55 to the rigid lower jaw member 52.

To achieve tissue cutting, a high voltage potential is created between the cut electrode 55 and the lower jaw member 52. This provides for the vaporization of tissue due to the heat created by local arcing around the cut electrode. As high temperature is encountered, the insulating material used to isolate the cut electrode in one embodiment withstands or operates well at high temperatures. Also, with the high voltage potential, the insulator in one embodiment has a high dielectric strength. The voltage potential is greater than 400 V-peak in order to achieve sufficient arcing. The actual potential however is directly related to the spacing between the cut electrode and the lower jaw member.

Arc suppression is another concern and as such quick rectification of RF waveform distortion due to arcing and/or limiting power output to prevent the degradation of the materials used in the construction of the jaw are provided. If an arc is allowed to persist for more than 100 micro seconds, there is an increased risk of device degradation. Also, due to the extreme heat associated with the localized arcing, RF energy application in accordance with various embodiments includes a predetermined duty cycle or a waveform with a high crest factor. It has been found that the crest factor associated with a sinusoidal waveform does not allow for a constant output without causing degradation to the device. Manipulation of the duty cycle or crest factor brings the average output power down over the entire activation of the device.

The electrosurgical instrument in accordance with various embodiments also fuses tissue using RF energy and in one embodiment only with the jaws in a fully open position, only utilizing the lower jaw, without cooperation of the upper jaw or tissue not captured between the upper and lower jaws but in contact with the lower jaw. FIG. 14 exemplarily illustrates a direction of current flow from the cut electrode 55 to the rigid lower jaw member 52.

In accordance with various embodiments, to cause tissue coagulation, a low voltage potential is maintained between the cut electrode 55 and the lower jaw member 52. The voltage potential is set to be lower than 100 V-peak in order to prevent localized arcing, but the actual potential is directly related to the spacing between the cut electrode and the lower jaw member. Tissue coagulation is caused by heat generated by the RF current between the two electrodes.

In one embodiment, the isolated wire 44 is routed to electrically couple the first jaw to the wiring harness in the actuator. The isolated wire extends from the distal end of the protective sleeve which is housed at the proximal end of the second jaw and extends into the first jaw. The first jaw can have a slot positioned to receive the isolated wire. The isolated wire then extends through a hole in the first jaw and drops into a slot in a nonconductive portion. The isolated wire then extends to the distal end of the nonconductive portion and drops through to the conductive pad.

In some embodiments, electrode geometry of or on the conductive pads of the jaws ensures that the sealing area completely encloses the distal portion of the cutting path. In accordance with various embodiments, the dimensions of the jaw surfaces are such that it is appropriately proportioned with regards to the optimal pressure applied to the tissue between the jaws for the potential force the force mechanism can create. Its surface area is also electrically significant with regards to the surface area contacting the tissue. This proportion of the surface area and the thickness of the tissue have been optimized with respect to its relationship to the electrical relative properties of the tissue.

In one embodiment, as illustrated in FIG. 15, an electrosurgical process such as a tissue fusion and/or cut process starts with depressing a switch on the tool (151), which starts an initial measurement sequence. With engagement of a switch on the tool, the generator takes initial measurements on the tissue (opens, shorts, etc.) (152) and based on the initial measurements initiates or does not initiate the supply of RF energy (153). In accordance with various embodiments, the generator measures tool and/or tissue impedance and/or resistance, and/or if a phase angle is within an acceptable range. In one embodiment, the generator performs a passive measurement of tissue between the electrodes of an electrosurgical tool connected to the generator utilizing RF energy with a low energy range (e.g., a voltage about 1-10 Volts) that does not cause a physiological effect. In various embodiments, the generator uses the initial impedance measurement to determine if the tool is shorted, faulty, open and the like. Based on a positive result of the initial check, the generator for example switches-in a supply of RF energy from the generator to the electrosurgical tool and ultimately to the tissue (154). After RF power is turned on and is being supplied continuously by the generator, the generator monitors the phase angle or difference and/or change of phase angle between current and voltage of the supplied RF energy (155).

At or upon a predefined or predetermined point, condition or threshold (156), the supply of RF energy is terminated (157). In this case, an acoustical and/or visual signal is provided indicating that the tissue is fused (or that an error has occurred (e.g., shorting of the electrodes) and/or an unexpected condition has occurred (e.g., permissible although unexpected switch release)). In accordance with various embodiments, the predefined point, condition or threshold and/or initialization checks are determined based on a tool algorithm or script provided for a connected electrosurgical tool, procedure or preference. In accordance with various embodiments, the product of measured tissue permittivity and conductivity or an initial phase shift is utilized to determine the end point for a connected tool.

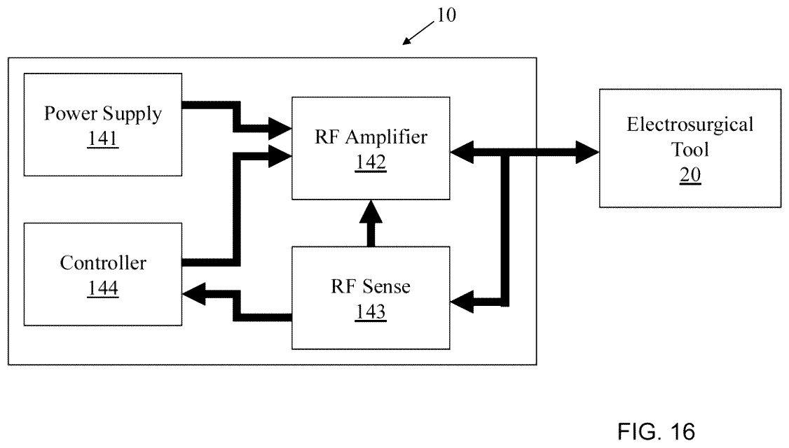

Referring now to FIG. 16, in one embodiment, the electrosurgical generator 10 is connected to AC main input and a power supply 141 converts the AC voltage from the AC main input to DC voltages for powering various circuitry of the generator. The power supply also supplies DC voltage to an RF amplifier 142 that generates RF energy. In one embodiment, the RF amplifier 142 converts 100 VDC from the power supply to a sinusoidal waveform with a frequency of 350 kHz which is delivered through a connected electrosurgical instrument. RF sense circuitry 143 measures/calculates voltage, current, power and phase at the output of the generator in which RF energy is supplied to a connected electrosurgical instrument 20. The measured/calculated information is supplied to a controller 144.

In one embodiment, the RF sense analyzes the measured AC voltage and current from the RF amplifier and generates DC signals for control signals including voltage, current, power, and phase that are sent to the controller for further processing. In one embodiment, RF sense 143 measures the output voltage and current and calculates the root means square (RMS) of the voltage and current, apparent power of the RF output energy and the phase angle between the voltage and current of the RF energy being supplied through a connected electrosurgical instrument. In particular, the voltage and current of the output RF energy are processed by analog circuitry of the RF sense to generate real and imaginary components of both voltage and current. These signals are processed by an field-programmable gate array (FPGA) to give different measurements relating to voltage and current, including the RMS measurements of the AC signals, phase shift between voltage and current, and power. Accordingly, in one embodiment, the output voltage and current are measured in analog, converted to digital, processed by an FPGA to calculate RMS voltage and current, apparent power and phase angle between voltage and current, and then are converted back to analog for the controller.

For each device port there are a pair of signals for voltage and a pair of signals for current that originate from the RF amplifier 142. In one embodiment, the generator has two redundant RF sense circuits 143a, 143b that measures voltage and current for each device at different locations on the RF amplifier. The first RF Sense circuit senses current by sense resistors delivered through a connected electrosurgical instrument on either device port 1 or device port 2, and the voltage measured across return to output on either device port 1 or device port 2. The second RF Sense circuit senses current by sense resistors, returned from a connected electrosurgical instrument on either device port 1 or device port 2, and the voltage 146a, 146b measured across output to return on either device port 1 or device port 2. The voltage input signals are high voltage sinusoidal waveforms at 350 kHz that are attenuated and AC coupled by a voltage divider and an inverting filter to remove DC bias on the signals. An inverting filter is used as the voltage and current inputs are 180 degrees out of phase as they are measured at opposite polarities. For each voltage input signal, two separate inverted and non-inverted voltage sense signals are generated. In one embodiment, a differential voltage measurement is made between the current input signals to generate two separate pairs of inverted and non-inverted current sense signals. The current input signals represent voltage across a shunt resistor on the RF Amplifier in which this voltage is proportional to the current flowing through the shunt resistor. The current input signals are low voltage sinusoidal waveforms at 350 kHz that are amplified using a non-inverting filter to remove DC bias on the signals. The RF Sense generates a signal that is analogous to multiplying each voltage and current signal by predetermined reference signals. As such, the RF Sense provides the non-inverted voltage and current sense signals when the waveform is positive, the inverted voltage and current sense signals when the waveform is negative, and a ground signal when the waveform is zero.

The RF sense in accordance with various embodiments receives four reference synchronization signals supplied by the controller via the RF amplifier. The synchronization signals are 350 kHz pulse signals with the same duty cycle but with differing phase shifts and in one embodiment are 90 degrees phase shifted from each other. Two of the synchronization signals are used to generate the in-phase waveforms to generate the real component of the input waveforms and the two other synchronization signals are used to generate the quadrature waveforms to generate the imaginary components of the input waveforms. These signals are processed further to generate control signals to a plurality of switches. The outputs of the switches are tied together to generate a single output. In one embodiment, the control signals to the switches determine which input signal passes through to the single output. In accordance with various embodiments, a first combination allows non-inverted voltage and current sense signals to pass through which represents or is analogous to multiplying these sense signals by a positive pulse. A second combination allows the inverted voltage and current sense signals to pass through which represents or is analogous to multiplying these sense signals by a negative pulse. A third combination allows the ground signal to pass through generating a zero voltage output which represents or is analogous to multiplying the sense signals by zero. Each output is supplied to a low pass filter that generates a DC voltage corresponding to the real or imaginary component of the sensed signals. These signals are supplied to ADCs which sends a digital signal to the FPGA.

In one embodiment, Controller 144 controls the RF amplifier 142 to affect the output RF energy. For example, Controller utilizes the information provided by the RF sense 143 to determine if RF energy should be outputted and when to terminate the output of RF energy. In one embodiment, the controller compares a predetermined phase threshold based on a particular tissue in contact with the connected electrosurgical device 20 to determine when to terminate the output of RF energy. In various embodiments, the controller performs a fusion process described in greater detail below and in some embodiments the controller receives the instructions and settings or script data to perform the fusion process from data transmitted from the electrosurgical instrument.

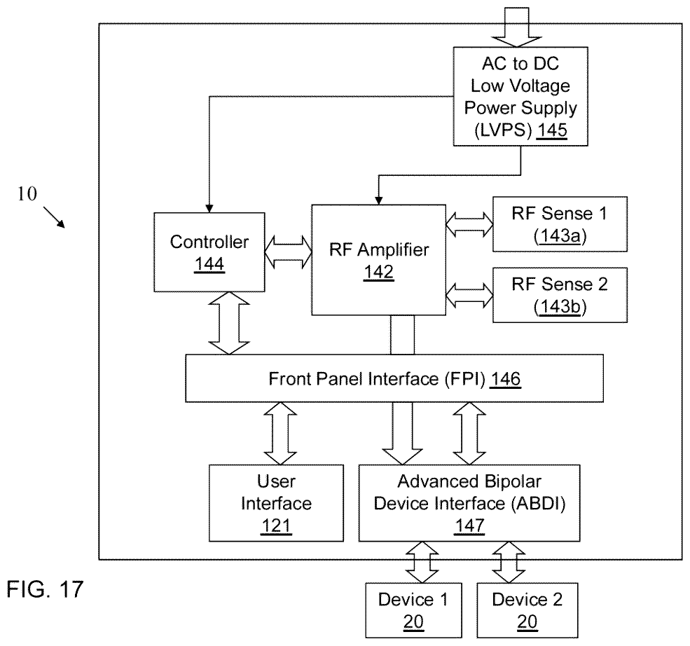

In accordance with various embodiments as shown in FIG. 17, the generator has six major sub-systems or modules of circuitry that include System Power or Power Supply 145, Controller 144, Front Panel Interface 146, Advanced Bipolar Device Interface 147, RF Amplifier 142 and RF Sense 143. In accordance with various embodiments, one or more of the circuitry may be combined or incorporated with other circuitry. The power supply 145 is configured to provide DC voltages to all the other circuitry or sub-systems along with control signals to control the power supply outputs. The power supply receives AC power input that is 90-264 VAC, 47-63 Hz and in one embodiment the power supply has a switch, integrated or separate, that is configured to connect or disconnect the AC power input from the generator. The controller through the Front Panel Interface (FPI) and Advanced Bipolar Device Interface (ABDI) supports the user interface 121 and instrument connections for electrosurgical devices 1 and 2 connected to the electrosurgical generator.

The RF Amplifier 142 generates high power RF energy to be passed through a connected electrosurgical instrument and in one example, an electrosurgical instrument for fusing of tissue. The RF Amplifier in accordance with various embodiments is configured to convert a 100 VDC power source to a high power sinusoidal waveform with a frequency of 350 kHz which is delivered through the ABDI 147 and eventually the connected electrosurgical device. The RF Sense 143 interprets the measured AC voltage and current from the RF amplifier 42 and generates DC control signals, including voltage, current, power, and phase, that is interpreted by Controller 144.

The generator has a plurality of specialized connection receptacles, in the illustrated embodiment device port 1 and device port 2, that are used only for connecting to advanced bipolar devices, such as an advanced bipolar electrosurgical instrument. The specialized receptacles each include an array spring-loaded probes or pogo pins. The generator in various embodiments includes a circuit to detect the presence of an advanced bipolar device prior to energizing any active output terminals at the receptacles.

The Front Panel Interface (FPI) 146 is configured to drive a display, device signals from the controllers and LED backlights for front panel buttons. The FPI is also configured to provide power isolation through regulators and provide functionality for the front panel switches/buttons. In one embodiment, the ABDI 147 is used as a pass-through connection which provides a connection to the devices through the FPI. The FPI also provides connection between Controller 144 and a connected electrosurgical device through the ABDI. The device interface in one embodiment is electrically isolated from the rest of the FPI. The interface in various embodiments includes lines that read and write to an ferromagnetic random access memory (FRAM) on an advanced bipolar device, read a trigger switch and/or read a signal that indicates a device is connected. In one embodiment, a device memory circuit is provided that utilizes the controller's serial peripheral interface (SPI) to read and write the FRAM of the advanced bipolar device. In one embodiment, the FRAM is replaced with a microcontroller and the interface includes an interrupt line so all information passed through a digital interface between the electrosurgical device and the generator. FPI provides isolation for SPI signals to and from advanced bipolar device through ABDI. In one embodiment, the SPI interface is shared between two advanced bipolar devices with port pins being used as chip selects.

In accordance with various embodiments, the generator includes a SPI communication bus that allows the controller to have bi-directional communication with complex programmable logic devices (CPLDs) and the RF Sense FPGAs. In various embodiments, the FPI provides SPI interface between the controller and connected devices through an ABDI connector to communicate with the FRAM on the advanced bipolar devices. FPI also provides electrical isolation for low voltage signals from between controller and the ABDI. The device interface on the ABDI is configured to transmit RF energy to the connected device along with SPI communication. In one embodiment, the ABDI connects a signal from a device that indicates it is connected.

The FPI-ABDI interface provides power to the devices that connect to the generator, SPI communication between controller and the devices, device switch signals from the devices to the controller, and device connected signals from the devices to the controller. ABDI provides the RF energy to each connected advanced bipolar device through a separate pogo pin array. The FPI provides signal, low voltage power and high voltage RF power from the FPI and RF Amplifier to the connected device through the ABDI connector via the pogo pin array.

In accordance with various embodiments, an operations engine enables the generator to be configurable to accommodate different operational scenarios including but not limited to different and numerous electrosurgical tools, surgical procedures and preferences. The operations engine receives and interprets data from an external source to specifically configure operation of the generator based on the received data.

The operations engine receives configuration data from a database script file that is read from a memory device on a device plug. The script defines the state logic used by the generator. Based on the state determined and measurements made by the generator, the script can define or set output levels as well as shutoff criteria. The script in one embodiment includes trigger events that include indications of short condition for example when a measured phase is greater than 70 degrees or an open condition for example when a measured phase is less than −50 degrees.

In one embodiment, the operations engine provides system states and user states. System states are predefined or predetermined states that control or manage specific predefined or predetermined operations of the generator, such as successfully applying RF energy or indicating an error. System states in one embodiment are a pre-defined set of configuration that the system can be in (e.g., idle vs. energized) and whose functions are hard-coded into the electrosurgical generator. For example, a RF Done state is a system state that indicates that an RF energy cycle has been completed without errors. User states provide a framework through which customized or specialized operations and values can be established by direction from an external source for a particular tool, procedure and/or preference.

In one embodiment, the script sets forth the system states and their exit conditions, e.g., expiration times or pointers or directions to another state and where the user states begin. For each user state, operation parameters for the specific state can be defined such as power, voltage, and current settings or are carried over from a previous state. In one embodiment, the user states may provide device, operator or procedural specific states and in one embodiment, the user states may be provided for testing or diagnostics specific states.

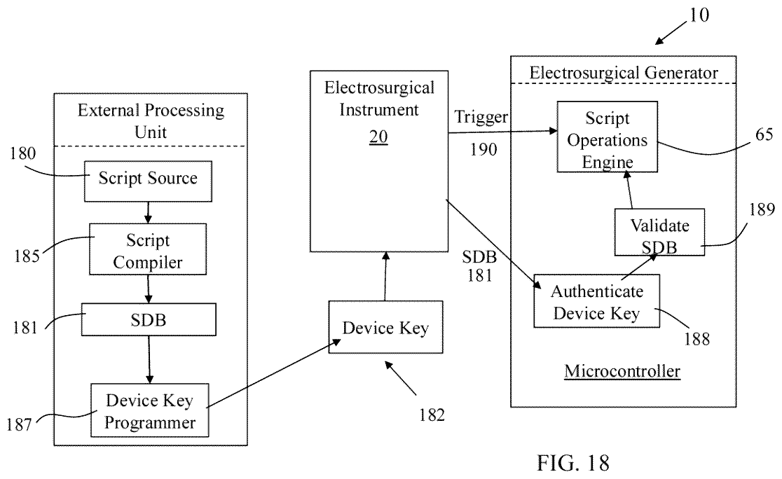

Referring to FIG. 18, the generator 10 receives script information from the electrosurgical device or instrument 20 when the device is connected. The generator uses this script information to define a number of states and the order of execution of the states.

The script source file or script information 180 written by the device script author and not resident on the instrument or the generator 10 is text or user readable. The script information is compiled using a script complier 185 to generate a device script database or binary file (SDB) 101. The script binary file is transferred by a device key programmer 187 to a memory module that is connectable or incorporated into the electrosurgical instrument 20 via a device key 182. As the electrosurgical instrument is connected to the electrosurgical generator, the generator authenticates the script binary file and/or the instrument (188). The generator validates the script binary file (189) and if validated the operations engine utilizes the script initiated by the actuation by the connected instrument (190). Script source file in one embodiment is a text file containing a device script that is specific for a specific electrosurgical instrument, generator and/or surgical procedure. The script source file for a device in one embodiment includes information containing parameters and a script (states, functions, events) for the electrosurgical generator and/or electrosurgical instrument. After successful validation, the script compiler assembles data into a binary format that defines a state machine for use by the electrosurgical generator. Script compiler as shown in FIG. 18 in one embodiment is separate from the electrosurgical generator and is responsible for reading in text from the script source file and validating its contents.

When the memory module is inserted into the generator, the generator downloads a binary file that is stored in a ferromagnetic random access memory (FRAM) or microcontroller disposed within the module. The binary includes logic for implementing the treatment algorithms or processes in accordance with various embodiments. The generator in various embodiments includes firmware/software, hardware or combinations thereof responsible for processing the binary to authentic the connected instrument and to execute the binary for performing the treatment algorithm. In this manner, the generator is configured to operate only with authenticated, compatible hand tools.

In one embodiment, instrument scripts or script database represent an instrument process for a specific or given instrument. The instrument scripts are stored on memory connected to or integrated with an instrument, the controller or a combination thereof. The event handler responds to specific events, such as a switch activation/de-activation, instrument positions or exceeding measurement thresholds. The operations engine based on the detected event if appropriate for a given event provides output to the connected instrument. In one embodiment, an event is a discrete change, as in a switch is asserted or de-asserted.

Script state is a block or set of script functions or operation conditions and script events or indicators. Script functions are configurable instructions for controlling the generator and/or the instruments. Script operators are logical and comparison operations performed during a script event evaluation. Script parameters are configuration data used by all states and events of a script and in one embodiment are declared in their own dedicated section of the script file. Script events are a discrete change in an electrosurgical generator measurement. When a Script Event occurs, for example, a sequence of script functions is executed.

In accordance with various embodiments, phase angle between voltage and current and/or the change or phase angle rate is utilized to maximize the amount of time the tissue is in a predetermined temperature range. In one embodiment, the predetermined temperature range is between 60 degrees C. to 100 degrees C. In one embodiment, low voltage is utilized to minimize temperature effects while accelerating sealing or fusing time.

In accordance with various embodiments, tissue is grasped between jaws of the bipolar electrosurgical device. The bipolar electrosurgical device removably connected to an electrosurgical generator is supplied RF energy that upon command is supplied to the tissue. The supplied RF energy has a predetermined voltage range that heats the tissue at a predetermined rate. In one embodiment, the predetermined voltage range is between 20 Vrms to 50 Vrms. During the application of RF energy, the phase angle between output voltage and current are monitored to identify phase increases or decreases. Initially, phase angle is monitored to determine a change in phase angle or rate from increasing to decreasing. It is contemplated that once this point of inflection has occurred, it is determined that water in the jaws of the device has reached 100 degrees C. and the temperature to cause the required tissue affect has been exceeded. A shutoff point is then determined to terminate the supply of RF energy.

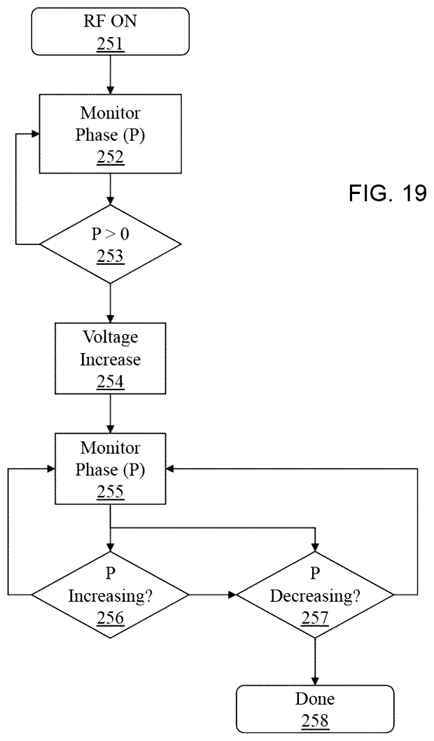

Exemplary RF energy control process for the electrosurgical generator and associated electrosurgical tool for fusing tissue in accordance with various embodiments are shown in FIGS. 19-21. In various embodiments, as illustrated in FIG. 19, RF energy is supplied by the generator through the connected electrosurgical tool (251). The generator monitors at least the phase and/or change/rate of phase of the supplied RF energy (252). If a phase/phase change is greater than zero or trending positive (253), voltage is increased (254). The generator continues to monitor at least the phase and/or change/rate of phase of the supplied RF energy (255). If the phase/phase change continues to increase (256), the generator continues to monitor the phase and/or change of phase. If the phase/phase change decreases (257), the process is done or termination procedures are initiated and/or RF energy supplied by the generator is stopped (258).

In one embodiment, prior to the start of the process, impedance is measured to determine a short or open condition through a low voltage measurement signal delivered to a connected electrosurgical tool. In one embodiment, passive impedance is measured to determine if the tissue grasped is within the operating range of the electrosurgical tool (2-200Ω). If the initial impedance check is passed, RF energy is supplied to the electrosurgical tool. After which impedance/resistance is not measured or ignored.

Initially, initial parameters are set to prepare for sealing of tissue grasped between the jaws. In one embodiment, voltage and current settings are set to a specific setting. In one embodiment, voltage of the RF energy is applied in a ramping fashion starting from 30% of a global setting or user selected level (e.g., 27.5-88V for level 1, 25.0-80V for level 2 and 22.5V-72V for level 3). The voltage DAC is set to 30% of the voltage setting which on level 2 (medium) is 25.5 Vrms. The phase is monitored to determine a phase angle above zero degrees and to heat the tissue and water between the jaws of the electrosurgical device at a predetermined slow rate.

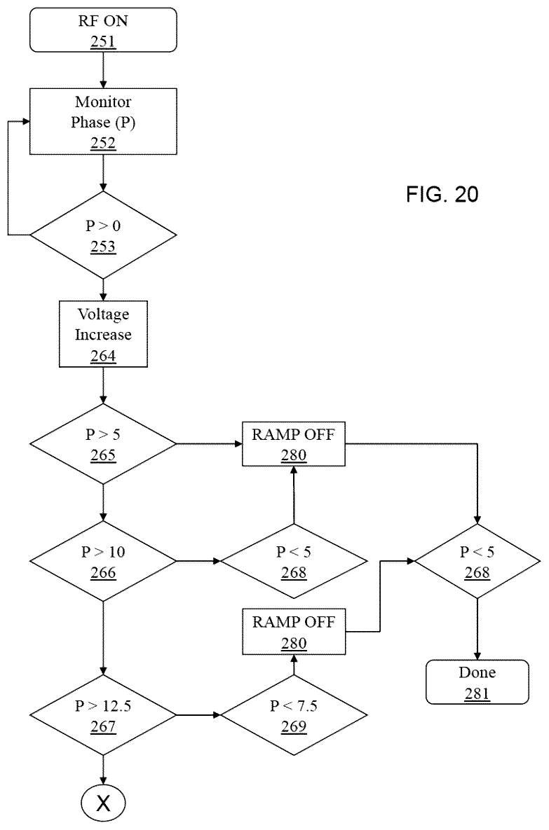

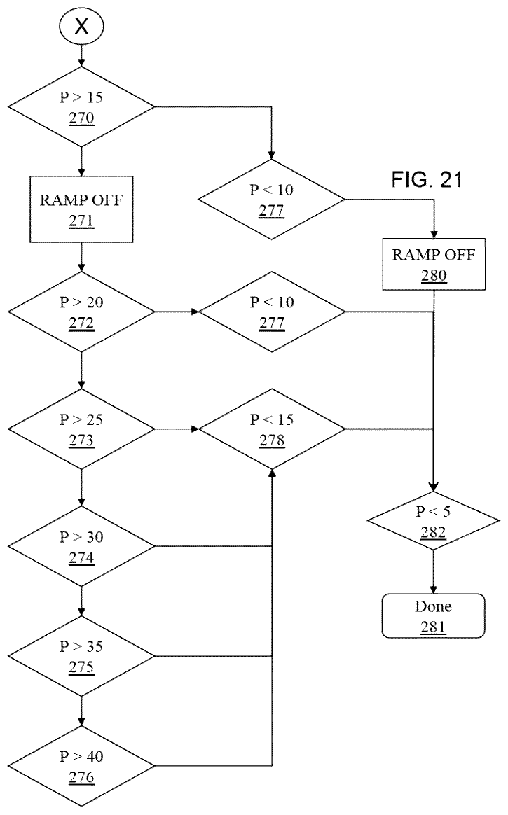

Referring now to FIGS. 20-21, in various embodiments, RF energy is supplied by the generator through the connected electrosurgical tool (251). The generator monitors at least the phase and/or change/rate of phase of the supplied RF energy (252). If a phase/phase change is greater than zero or trending positive (253), voltage is increased (254). In various embodiments, voltage is increased at a predetermined rate, for example, 50% over 5 seconds, which is 42.5 Vrms on level 2 (medium) after the phase has increased to be greater than zero degrees. The ramping continues until a predetermined condition is met. In one embodiment, the ramping continues as the monitored phase angle increases to above five degrees. This ensures that phase is increasing as expected based on heating of the tissue.

In a next subsequent state (265), if the monitored phase angle increases above a predetermined phase value, e.g., 5 degrees, phase continues to be monitored for an increasing state or condition. This is contemplated that such an increasing condition is an indication that the temperature of the tissue and water between the jaws is increasing but less than 100 degrees C. However, the monitored phase indicating a decreasing state or condition provides a different indication. It is contemplated that such an indication is that the temperature of the tissue and water between the jaws has reached at least 100 degrees C. and/or the desired tissue affect has been completed, e.g., sealing and/or cutting of the tissue.

In one or more subsequent states, the monitored phase angle is checked for an increasing or decreasing condition. In accordance with various embodiments, various incremental or periodic checks are performed along with various incremental or periodic updates to various predetermined thresholds or indications to determine an increasing phase angle or rates of change condition or a decreasing phase angle or rates of change condition.

When one or more of the subsequent states determines that the phase angle, rate or trend of the phase angle is decreasing rather than increasing, the indication is that the desired tissue affect has been completed, e.g., sealing and/or cutting of the tissue. As such, it is contemplated that in such an indication the temperature of the tissue and water between the jaws has reached at least 100 degrees C.

At such a state or in a subsequent state, voltage ramping or increasing of the output RF energy is ramped down or decreased. As such, the rapid boiling of water is reduced or prevented and temperature is held steady or constant until a predetermined condition is reached. In one embodiment, the predetermined condition is the phase angle dropping to at least 5 degrees.

In accordance with various embodiments, in a next subsequent state (266), if the monitored phase angle increases above a predetermined phase value, e.g., 10 degrees, phase continues to be monitored for an increasing state or condition. If however the monitored phase angle instead indicates a decreasing state, e.g., decreases below a predetermined phase value, e.g., five degrees (268) or a predetermined time limit has been reached, voltage increase is stopped (280). Subsequently, if the monitored phase angle continues to indicate a decreasing state, e.g., decreases below a predetermined phase value, e.g., five degrees (268) or if a predetermined time limit has been reached, RF energy is stopped and the process ends (281).

If the monitored phase angle indicates an increasing state, e.g., in a next subsequent state (267), if the monitored phase angle increases above a predetermined phase value, e.g., 12.5 degrees, phase continues to be monitored for a continued increasing state or condition. If the monitored phase angle instead now indicates a decreasing state, e.g., decreases below a predetermined phase value, e.g., 7.5 degrees (269) or a predetermined time limit has been reached, voltage increase is stopped (280). Subsequently, if the monitored phase angle continues to indicate a decreasing state, e.g., decreases below a predetermined phase value, e.g., five degrees (268) or if a predetermined time limit has been reached, RF energy is stopped and the process ends (281).

In a next subsequent state (270), if the monitored phase angle indicates an increasing state, e.g., increases above a predetermined phase value, e.g., 15 degrees, voltage increase is stopped (271) and phase continues to be monitored for an increasing state or condition. If however the monitored phase angle instead indicates a decreasing state, e.g., decreases below a predetermined phase value, e.g., ten degrees (277) or a predetermined time limit has been reached, voltage increase is stopped (280). Subsequently, if the monitored phase angle continues to indicate a decreasing state, e.g., decreases below a predetermined phase value, e.g., five degrees (282) or if a predetermined time limit has been reached, RF energy is stopped and the process ends (281). In a next subsequent state (272), if the monitored phase angle indicates an increasing state, e.g., increases above a predetermined phase value, e.g., 20 degrees, phase continues to be monitored for an increasing state or condition. If the monitored phase angle instead indicates an decreasing state, e.g., decreases below a predetermined phase value, e.g., ten degrees (277) or a predetermined time limit has been reached, phase continues to be monitored for a decreasing state or condition. Subsequently, if the monitored phase angle decreases below a predetermined phase value, e.g., five degrees (282) or if a predetermined time limit has been reached, RF energy is stopped and the process ends (281).

In a next subsequent state (273), if the monitored phase angle continues to indicate an increasing state, e.g., increases above a predetermined phase value, e.g., 25 degrees, phase continues to be monitored for an increasing state or condition. If the monitored phase angle decreases instead below a predetermined phase value, e.g., fifteen degrees (278) or a predetermined time limit has been reached, phase continues to be monitored for a decreasing state or condition. Subsequently, if the monitored phase angle decreases below a predetermined phase value, e.g., five degrees (282) or if a predetermined time limit has been reached, RF energy is stopped and the process ends (281). In a next subsequent state (274), if the monitored phase angle increases above a predetermined phase value, e.g., 30 degrees, phase continues to be monitored for an increasing state or condition. If the monitored phase angle instead decreases below a predetermined phase value, e.g., fifteen degrees (278) or a predetermined time limit has been reached, phase continues to be monitored for a decreasing state or condition. Subsequently, if the monitored phase angle decreases below a predetermined phase value, e.g., five degrees (285) or if a predetermined time limit has been reached, RF energy is stopped and the process ends (281).

In a next subsequent state (275), if the monitored phase angle increases above a predetermined phase value, e.g., 35 degrees, phase continues to be monitored for an increasing state or condition. However, if the monitored phase angle instead decreases below a predetermined phase value, e.g., fifteen degrees (278) or a predetermined time limit has been reached, phase continues to be monitored for a decreasing state or condition. Subsequently, if the monitored phase angle decreases below five degrees (282) or if a predetermined time limit has been reached, RF energy is stopped and the process ends (281). In a next subsequent state (276), if the monitored phase angle increases above a predetermined phase value, e.g., 40 degrees, phase is monitored for a decreasing condition and if the monitored phase angle decreases below a predetermined phase value, e.g., fifteen degrees (278) or a predetermined time limit has been reached, phase continues to be monitored for a decreasing state or condition. Subsequently, if the monitored phase angle decreases below a predetermined phase value, e.g., five degrees (282) or if a predetermined time limit has been reached, RF energy is stopped and the process ends (281). It is contemplated and noted that, for the exemplary and operational seal/fuse and cut/dissection process or system provided above and throughout the application, the frequency of incremental checks and/or the indications of increasing or decreasing states, e.g., predetermined angles or rates of change, can vary to provide different and various levels and granularity of regulation or control as desired or required based on the specific electrosurgical instrument, generator, tissue and/or surgical procedure.

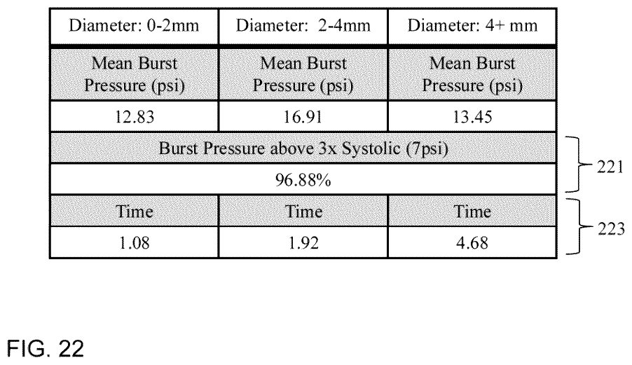

FIGS. 22-24 are graphical representations of exemplary vessel sealing/fusing utilizing systems and processes in accordance with various embodiments of the present invention. As shown, the success rate 223 of providing a tissue seal above 3× systolic burst pressure are high and time 223 for sealing of vessels up to 4 mm in size was small, e.g., less than 2 seconds. The times for sealing vessels between 4-7 mm was also reduced, e.g., less than 5 seconds. The time for the phase angle to decrease from the maximum to a predetermined phase value, e.g., 5 degrees, is longer than those up to 4 mm. Such changes or reduction in sealing time while also providing successful vessel seals, e.g., withstanding above 3× systolic burst pressure, in accordance with various embodiments is refined by identifying and/or triggering at the point of inflection of the derivate of the phase trend and incorporated into incremental checks, state indicators or thresholds. In various embodiments, the point of inflection of the derivate of the phase trend is identified at the point where the phase trend changes from an increasing state to a decreasing state.

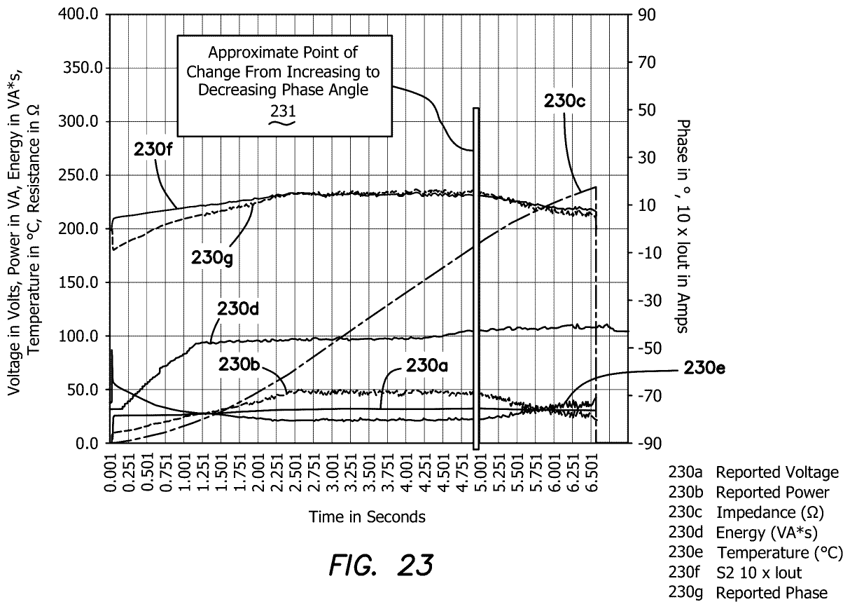

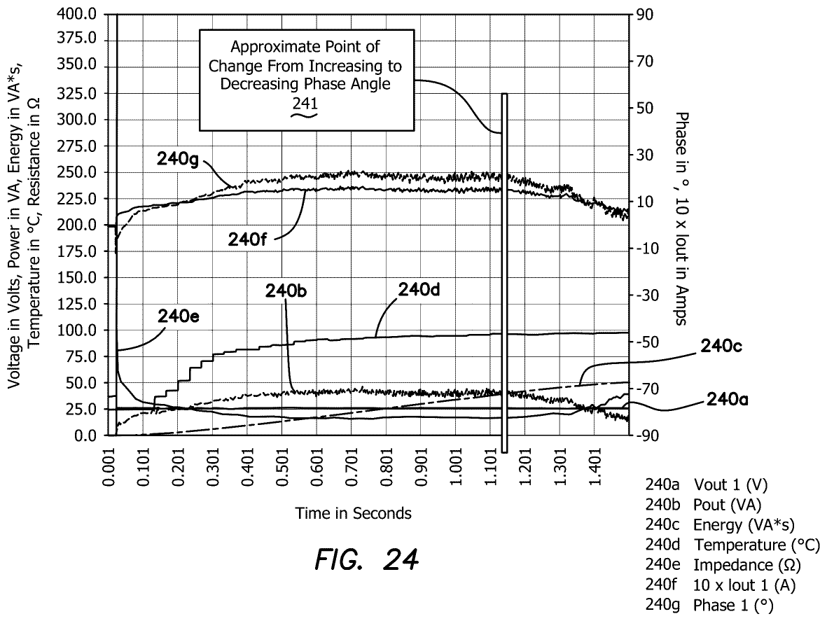

As shown in FIG. 23, the vessel was 6.62 mm in diameter and was successfully sealed, e.g., having a burst pressure of 12.7 psi. Additionally, as shown, the phase angle 230g increases as RF energy is applied. The rate of the increase is not rapid but rather sufficiently slow as well as the temperature 230d of the tissue. The point of inflection 231, e.g., the point at which phase changes from increasing to decreasing occurs approximately 1.5 seconds before RF energy is stopped. As shown in FIG. 24, the vessel was 1.89 mm in diameter and was successfully sealed, e.g., having a burst pressure of 13 psi. The overall trend of phase angle 240g and temperature 240d is similar as with the previous vessel seal, although the time scale shown in FIG. 24 is approximately ¼ of that of FIG. 23. Also, as illustrated, phase 230g, 240g are shown relative to other tissue readings or indicators such as voltage 230a, 240a; power 230b, 240b; impedance 230e, 240e; energy 230c, 240c; temperature 230d, 240d; and current 230f, 240f. Additionally, although shown in FIGS. 23-24, in various embodiments, the generator is configured to not measure or calculate one or more of the indicators or readings, e.g., temperature or energy, to reduce operational and power costs and consumptions and the number of parts for the generator. The additional information or readings are generally provided or shown for context purposes.

It is noted that the impedance of the tissue is near its minimum for the entire seal cycle. As such, this provides a low voltage and high current and thus consistent power delivery throughout the seal cycle. Efficient or consistent power delivery reduces thermal spread. In accordance with various embodiments, the time for sealing can be decreased, reduction in voltage output to below 50 Vrms and/or reduction of power output to below 50 Watts. To avoid false readings, in accordance with various embodiments, the electrosurgical generator does not measure resistance or impedance of the tissue during the supply of RF energy to the tissue.

In accordance with various embodiments, an electrosurgical system is provided that decreases thermal spread, provides lower output levels and efficient power delivery for sealing vessels or tissue in contact with a bipolar electrosurgical instrument through the controlled and efficient supply of RF energy.

As described throughout the application, the electrosurgical generator ultimately supplies RF energy to a connected electrosurgical instrument. The electrosurgical generator ensures that the supplied RF energy does not exceed specified parameters and detects faults or error conditions. In various embodiments, an electrosurgical instrument provides the commands or logic used to appropriately apply RF energy for a surgical procedure. An electrosurgical instrument for example includes memory having commands and parameters that dictate the operation of the instrument in conjunction with the electrosurgical generator. For example, in a simple case, the generator can supply the RF energy but the connected instrument decides how much or how long energy is applied. The generator however does not allow the supply of RF energy to exceed a set threshold even if directed to by the connected instrument thereby providing a check or assurance against a faulty instrument command.

Turning now to some of the operational aspects of the electrosurgical tool or instrument described herein in accordance with various embodiments, once a vessel or tissue bundle has been identified for fusing, dissecting or both, the first and second jaws are placed around the tissue. The movable handle 23 is squeezed moving the movable handle proximally with respect to the stationary housing 28. As the movable handle moves proximally the first jaw pivots towards the second jaw effectively clamping the tissue. Radio frequency energy is applied to the tissue by depressing the activation button on the stationary handle. Once the tissue has been fused, dissected or both, the movable handle is re-opened.

Alternatively or additionally, with the jaws in a fully open position or in an intermediate position between a fully open position and the engaged position, radio frequency energy can applied to the tissue in contact with a lower surface or portion of the lower jaw by depressing the activation button or a separate activation button to fuse and/or dissect tissue.

As described generally above and described in further detail below, various electrosurgical instruments, tools or devices can be used in the electrosurgical systems described herein. For example, electrosurgical graspers, scissors, tweezers, probes, needles, and other instruments incorporating one, some, or all of the aspects discussed herein can provide various advantages in an electrosurgical system. Various electrosurgical instruments and generator embodiments and combinations thereof are discussed throughout the application. It is contemplated that one, some, or all of the features discussed generally throughout the application can be included in any of the embodiments of the instruments, generators and combinations thereof discussed herein. For example, it can be desirable that each of the instruments described include a memory for interaction with the generator as previously described and vice versa. However, in other embodiments, the instruments and/or generators described can be configured to interact with a standard bipolar radio frequency power source without interaction of an instrument memory. Further, although various embodiments may be described in terms of modules and/or blocks to facilitate description, such modules and/or blocks may be implemented by one or more hardware components, e.g., processors, Digital Signal Processors (DSPs), Programmable Logic Devices (PLDs), Application Specific Integrated Circuits (ASICs), circuits, registers and/or software components, e.g., programs, subroutines, logic and/or combinations of hardware and software components. Likewise, such software components may be interchanged with hardware components or a combination thereof and vice versa.

Further examples of the electrosurgical unit, instruments and connections there between and operations and/or functionalities thereof are described in U.S. patent application Ser. No. 12/416,668, filed Apr. 1, 2009, entitled “Electrosurgical System”; Ser. No. 12/416,751, filed Apr. 1, 2009, entitled “Electrosurgical System”; Ser. No. 12/416,695, filed Apr. 1, 2009, entitled “Electrosurgical System”; Ser. No. 12/416,765, filed Apr. 1, 2009, entitled “Electrosurgical System”; and Ser. No. 12/416,128, filed Mar. 31, 2009, entitled “Electrosurgical System”; the entire disclosures of which are hereby incorporated by reference as if set in full herein. Certain aspects of these electrosurgical generators, tools and systems are discussed herein, and additional details and examples with respect to various embodiments are described in U.S. Provisional Application No. 61/994,215, filed May 16, 2014, entitled “Electrosurgical Fusion Device”; 61/944,185, filed May 16, 2014, “Electrosurgical Generator with Synchronous Detector”; 61/994,415, filed May 16, 2014, “Electrosurgical System”; and 61/944,192, filed May 16, 2014, entitled “Electrosurgical Generator”, the entire disclosures of which are hereby incorporated by reference as if set in full herein.

The above description is provided to enable any person skilled in the art to make and use the surgical instruments and perform the methods described herein and sets forth the best modes contemplated by the inventors of carrying out their inventions. Various modifications, however, will remain apparent to those skilled in the art. It is contemplated that these modifications are within the scope of the present disclosure. Additionally, different embodiments or aspects of such embodiments may be shown in various figures and described throughout the specification. However, it should be noted that although shown or described separately each embodiment and aspects thereof may be combined with one or more of the other embodiments and aspects thereof unless expressly stated otherwise. It is merely for easing readability of the specification that each combination is not expressly set forth. Also, embodiments of the present invention should be considered in all respects as illustrative and not restrictive.

1. An electrosurgical system comprising:
an electrosurgical instrument comprising:
a first jaw comprising a first electrode; and
a second jaw opposing the first jaw and comprising a second electrode, the first and second jaws pivotably arranged to grasp tissue between the first and second jaws and the first and second electrodes of the first and second jaws being arranged to transmit radio frequency energy (RF) between the first and second electrodes;
an electrosurgical generator arranged to supply the radio frequency (RF) energy, the electrosurgical generator comprising a controller arranged to periodically monitor a phase angle of the supplied RF energy and configured to signal an RF amplifier to increase voltage of the supplied RF energy when the monitored phase angle is greater than zero and increasing.
